(19) United States
(12) Patent Application Publication
(10) Pub. No.: US 2013/0103058 A1
(43) Pub. Date: Apr. 25, 2013

(54) IMPLANTABLE HERNIA REPAIR DEVICE WITH A REMOVABLE MEMBER

(71) Applicant: Maher Gobran, Irvine, CA (US)

(72) Inventor: Maher Gobran, Irvine, CA (US)

(21) Appl. No.: 13/279,101

(22) Filed: Oct. 21, 2011

(51) Int. Cl. A61B 17/03

(52) U.S. Cl. 606/151

(57) ABSTRACT

The invention relates to implantable medical devices, in particular to implantable medical devices used in surgical procedures for hernia repairs.

The present invention relates to disposable medical devices. More specifically, the invention is directed to a medical device for facilitating hernia repair. The device of the present invention comprises two components—one is an implantable mesh-like member that is used to repair hernia damage and another is a resilient member that is used for facilitating the repair procedure and is extracted before the repair procedure is completed.

### BACKGROUND

Hernias are caused by defects in the muscle layer of the abdomen. Historically, attempts to reconstruct the abdominal wall muscles have been associated with a high recurrence rate.

Implantable devices for repairing hernia have been known for many years. They are used to repair damaged tissue and to provide structure for supporting surrounding tissue.

The most basic form of devices that can be used for hernia repair is a piece of mesh or any other flexible flat material that is strong enough to be affixed to the surrounding damaged tissue.

Over the past few decades, several medical devices comprising various meshes have been introduced for patching hernia defects. As a result, tension free repair has become possible, and the recurrence rate has decreased.

Despite the improvements associated with the introduction of such medical devices, mesh placement is associated with a set of complications. Some of such complications are: infection, mesh erosion into surrounding bowels, and adhesions.

Surgeons face a number of challenges while performing ventral hernia repairs. Some of such challenges are freeing adherent scarred bowel from the fascia, protecting the bowel during surgery, dealing with surrounding attenuated tissues, and keeping the mesh device flat until it is secured in place.

The key objective of a hernia repair procedure is to patch the hernia defect and to reinforce the surrounding weak muscle layer.

A number of mesh designs and techniques have been introduced to allow surgeons improve the outcome of hernia repair procedures and to avoid some of the common complications.

A number of mesh designs are available to surgeons. They are often categorized into non-absorbable and absorbable.

Non absorbable meshes are synthetic. They can be either knitted or smooth layer. Some of the materials in this category are Polytetrafluorethylene (PTFE), Polyester and Prolene.

Non absorbable meshes offer stronger, more permanent repair. However, they may be more susceptible to infection, causing a foreign body reaction and they are more prone to encapsulation, adhesions and erosion into surrounding bowel.

PTFE is characterized by the advantage of having smooth non porous surface, which limits its adhesions to bowel. However, it gets encapsulated, does not integrate itself into the surrounding tissues and, because of its non porous nature, it does not resist infection effectively.

Prolene meshes come in the form of knitted layer, which allows for granulation tissue to grow. Therefore, Prolene meshes are characterized by better resistance to infection. However, they tend to create strong adhesions to the bowel, and may get encapsulated. The currently available newer Prolene meshes are made of light weighted material (such as Proceed, Ultrapro and Physiomesh), coated by an absorbable layer that limits the risk of adhesions and foreign body reaction. These new designs appear to cause fewer complications than the older heavy non-coated ones. However because of their light weight, it is difficult to keep them flat in the surgical field.

Polyester meshes are light weight, fabric-like with braided polyester filaments. They incorporate better in the surrounding tissues. However, they are notorious of having a higher infection rate. In addition, it is very hard to keep them flat during surgery.

Hybrid designs, such as the Composix mesh with an inner PTFE and outer Prolene layers have been introduced. Such hybrid designs are characterized the smooth inner PTFE layer facing the bowel and the rough knitted Prolen layer facing the muscle layer.

Absorbable meshes are usually made of cadaveric, porcine or bovine de-epithelialized skin.

Absorbable meshes have a lower infection rate, hardly create any adhesions, or erosions into bowel, which make them safe alternatives to synthetic meshes. However, because they tend to get absorbed, they have a higher recurrence rate then synthetic meshes. They are more costly than synthetic meshes.

Light weighted synthetic meshes have been becoming very popular because they offer a permanent strong repair, with a lower complication rate than the old heavy designs.

The major challenge presented by light weighted meshes is the difficulty maintaining the mesh flat in the field, until it is secured to the surrounding muscles.

To overcome the challenge of keeping the mesh flat in the field, a composite mesh device with a rigid ring around its periphery has been suggested (Composix Kugel Mesh). The rigid ring around the periphery of the mesh device is intended to keep the mesh stretched and flat in the field. However, this design may cause additional complications. For example, with larger size meshes, the ring may get detached from the mesh and stab the bowels.

Ventral hernia repairs can be performed through two approaches: laparoscopic or open. With the open approach, the mesh can be placed either anterior to the fascia or in the properitoneal space posterior the fascia. Laparoscopically, the mesh can only be placed posterior to the fascia.

The properitoneal repair has a major advantage over anterior repair because it decreases the recurrence rate, as the mesh is being protected from the pushing intra-abdominal pressure by the overlying muscle layer.

Placing the mesh anterior to the muscle layer has the advantage of protecting the bowel from adhesions, with all the possible complications of erosion into bowel.

Because of all these factors, proper selection of the mesh material is critical for ensuring proper placement of the mesh device as well as for decreasing recurrence and complications rate.

### SUMMARY

The following is a summary description of illustrative embodiments of the invention. It is provided as a preface to assist those skilled in the art to more rapidly assimilate the detailed discussion, which ensues, and is not intended in any way to limit the scope of the claims, which are appended hereto in order to particularly point out the invention.

As discussed above, during a surgical procedure for hernia repair, it is important for a surgeon to be able to better keep the mesh unfolded and straight in the field until it is secured in place. This is more challenging with the growing trend of using light weighted mesh devices for hernia repair. An even more important objective during such procedures is to protect the surrounding bowels from accidental stabbing by the surgical needles while securing the mesh device in place.

The present invention is directed to a disposable medical device for the repair and reconstruction of injured tissue. The proposed device may be used in surgical procedures to repair hernias and other similar problems that require that a surgeon place and fix a patch at the repair site.

The preferred embodiment of a medical device according to the present invention consists of a flat mesh material that contains a pocket-like arrangement on one side of the mesh around the periphery of the mesh material and a relatively more resilient member inserted into the pocket-like arrangement to permit the surgeon to more easily place and maintain the mesh device in the field during the surgical procedure. When the mesh material is secured, the surgeon can remove the more resilient member from the pocket-like arrangement before the hernia repair procedure is completed.

### BRIEF DESCRIPTION OF THE DRAWINGS

FIG. 1 depicts a medical device pursuant to one embodiment of the present invention, with a pocket-like arrangement around the periphery of the device.

FIG. 2 depicts an alternative embodiment of the medical device according to the present invention, with a double-layer of mesh and a slit in the upper mesh layer.

### DETAILED DESCRIPTION

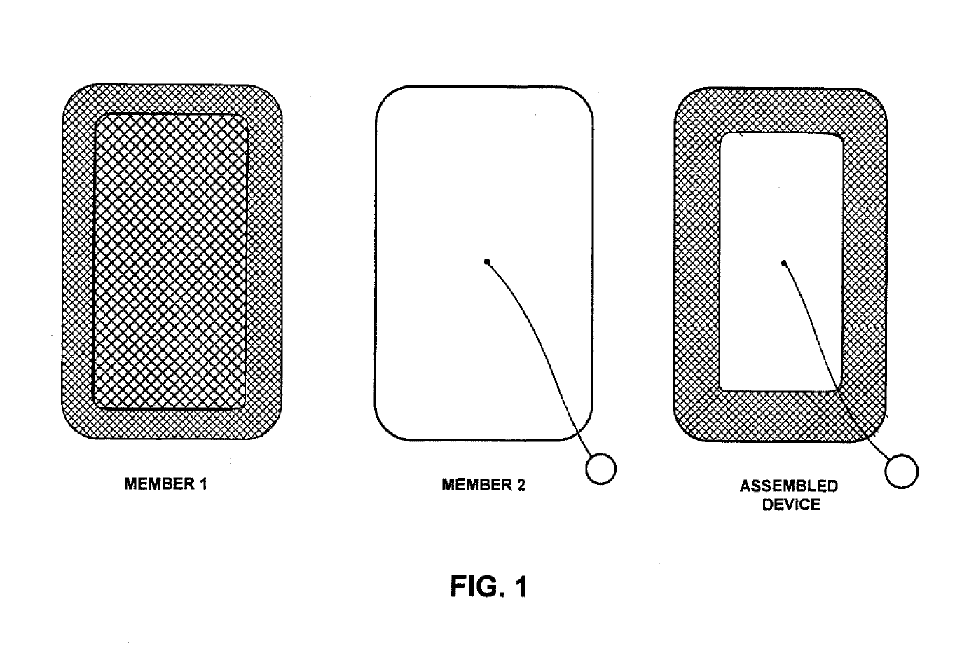

One embodiment of a medical device according to the present invention is shown in FIG. 1. The device consists of two members—planar mesh member 1 and resilient member 2. Planar mesh member 1 contains a pocket-like arrangement on one flat side along the periphery. Resilient member 2 is preferably characterized by a lesser area and is sized so that it can be securely placed within the pocket-like arrangement.

During a hernia repair surgery, the surgeon places member 1 together with resilient member 2 inserted within the pocket-like arrangement in the field.

Resilient member 2 causes the assembled medical device according to the present invention to be rigid enough to allow its secure placement in the field while the surgeon uses the outer area of planar mesh member 1 to securely affix planar mesh member 1 to the tissue surrounding the damaged area.

When the surgeon completes affixing planar mesh member 1, the surgeon can pull resilient member 2 out of the pocket-like arrangement, while leaving planar mesh member in place.

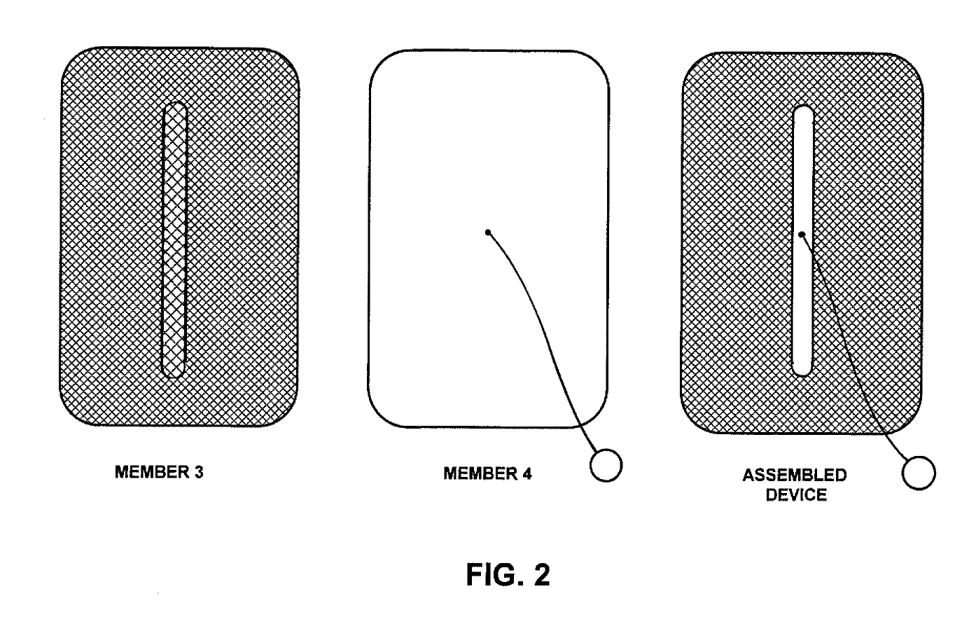

FIG. 2 shows an alternative embodiment of the medical device according to the present invention. In this embodiment, a two-layer planar mesh member 3 contains a slit in the upper mesh layer. Resilient member 5 is preferably characterized by a lesser area and is sized so that it can be inserted through the slit and thereby securely attached to two-layer planar mesh member 4.

During the surgical procedure, the surgeon uses the outer areas of two-layer planar mesh member 3 to attach it to the surrounding damaged tissue, after which the surgeon can pull resilient member 4 out of two-layer planar mesh member 3, thereby leaving only two-layer planar member 3 in the site of the hernia being repaired.

An abdominal retractor commonly used in abdominal surgery and known as a “fish,” may be used as resilient member 2 in the device depicted in FIG. 1 and as resilient member 4 in the device depicted in FIG. 2. The disposable abdominal retractor can be made of rubber or rubber-like material to be flexible enough to be folded and plied.

In one alternative embodiment of the present invention, the “fish,” or the resilient member, may be equipped with a cord, which the surgeon can use to pull on at the end of the closure to retrieve such resilient member.

The resilient member or retractor inserted inside the planar mesh member of the device according to the present invention serves to keep the planar mesh member unfolded and stable in a planar layer to facilitate the surgeon's ability to secure the planar mesh member and, thereby, repair the damaged hernia area.

At the same time, the resilient member protects the protruding bowels from stabbing while the planar mesh member is being fixed in place.

The use of the device according to this invention allows the attenuated fascia to overlap the planar mesh member and to be sutured to the upper layer of the planar mesh member towards both center and the periphery. As a result, the planar mesh member can be securely aligned in place, with the bowel being protected from needle stabbing.

A device according to the present invention may contain an unobstructed outer border area that can be used by a surgeon for placing sutures or staples to repair the damaged surrounding tissue.

Each embodiment of the invention described herein may be covered or impregnated with one or more antimicrobial agents.

Although embodiments of the device according to the present invention are described to have planar mesh members, the same invention may be implemented with planar members made of various currently known and future woven and non-woven materials. Some of such materials that are currently available are polypropylene, polyethylene, nylon and polytetrafluroethylene and other materials.

The resilient members according to the present invention may be made of rubber as well as any of the many currently available and future resilient materials, such as various silicone-based materials.

Other features of the present invention will become apparent from the following detail description of the invention when taken in connection with the accompanying drawings. It is understood that the drawings are designed for the purpose of illustration and are not intended as a definition of the limits of or restrictions on the present invention.

While the disclosure above sets forth the principles of the present invention, with the examples given for illustration only, one should realize that the use of the present invention includes all usual variations, adaptations and/or modifications, within the scope of the claims that follow as well as equivalents thereof.

Those skilled in the art will appreciate from the foregoing that various adaptations and modifications of the just described embodiments can be configured without departing from the scope and sprit of the invention. Therefore, it is to be understood that, within the scope of the appended claims, the invention may be practiced other than as specifically described herein.

What is claimed is:

1. A medical device comprising:
a planar mesh member with a first side and a second side;
a pocket-like arrangement located on said first side along the periphery of said planar mesh member; and
a resilient member inserted into said pocket-like arrangement.

2. The medical device of claim 1, whereby said resilient member is made of rubber.

3. The medical device of claim 1, whereby said resilient member contains a cord exposed when said resilient member is inserted into said pocket-like arrangement.

4. A medical device comprising:
a planar dual-layer mesh member with a first flat side and a second flat side;
a slit in said first flat side; and
a resilient member inserted into said slit.

5. The medical device of claim 4, whereby said resilient member is made of rubber.

6. The medical device of claim 4, whereby said resilient member contains a cord exposed when said resilient member is inserted into said slit.
